(19) United States
(12) Patent Application Publication
(10) Pub. No.: US 2021/0300917 A1
(43) Pub. Date: Sep. 30, 2021

(54) Solid State Forms of an Apoptosis-Inducing Agent and Processes Thereof

(71) Applicant: Lupin Limited, Mumbai (IN)

(72) Inventors: Lalitkumar Dilipsing Rajput, Pune (IN); Vasant Chhabu Vyavhare, Pune (IN); Radhakrishna Bhikaji Shivdavkar, Pune (IN); Yuvraj Dadasaheb Sudrik, Pune (IN); Rangan Mitra, Pune (IN); Sangram Gokhale, Pune (IN); Sunilkumar Vinubhai Gohel, Pune (IN); Rajinder Singh Siyan, Pune (IN); Nandu Baban Bhise, Pune (IN); Girij Pal Singh, Pune (IN)

(21) Appl. No.: 16/621,036

(22) PCT Filed: Jun. 9, 2018
(86) PCT No.: PCT/IB2018/054184

§ 371 Date: Dec. 10, 2019

(30) Foreign Application Priority Data
Jun. 10, 2017 (IN) 201721020367
Sep. 19, 2017 (IN) 201721033217
Mar. 7, 2018 (IN) 201821008393

(51) Int. Cl. C07D 471/04

(52) U.S. Cl. CPC C07D 471/04; C07B 2200/13

(57) ABSTRACT

Various crystalline and amorphous solid state forms of an apoptosis-inducing agent and preparations thereof are disclosed. Also discloses the use of crystalline forms for the preparation of amorphous form of apoptosis-inducing agent. Further discloses a process for preparation of the apoptosis-inducing agent. (Formula I)

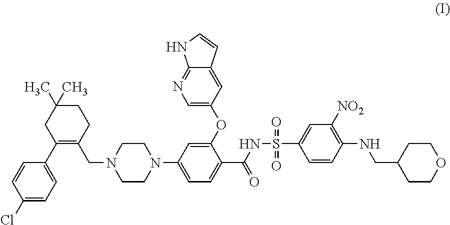 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

### FIELD OF INVENTION

The present invention relates to novel crystalline and amorphous solid state forms of an apoptosis-inducing agent and preparations thereof. The present invention further relates to the use of crystalline forms for the preparation of amorphous form of apoptosis-inducing agent. The present invention also relates to a process for preparation of the apoptosis-inducing agent.

### BACKGROUND OF THE INVENTION

Venetoclax, an apoptosis-inducing agent, referred to herein as compound of formula (I), has the systematic name 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridine-5-yloxy)benzamide, is a potent and selective Bcl-2 inhibitor having, inter alia, antitumor activity:

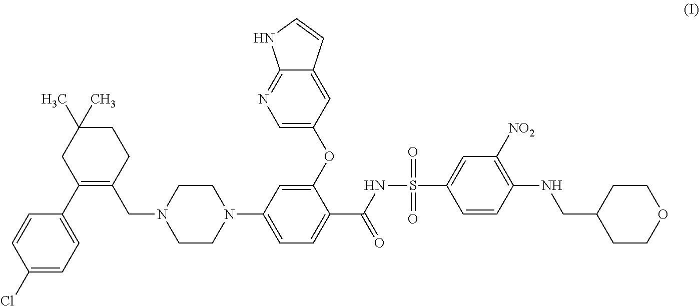 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

The U.S. Pat. No. 8,546,399 B2 describes Venetoclax, process for its preparation and its pharmaceutical compositions.

The U.S. Pat. No. 8,722,657 B2 describes crystalline forms of Venetoclax viz. free base anhydrate (pattern A), free base anhydrate (pattern B), free base hydrate (pattern C), free base hydrate (pattern D). This patent also describes dichloromethane solvate (pattern E), ethyl acetate solvate (pattern F and pattern G), acetonitrile solvate (pattern H and pattern I), acetone solvate (pattern J), hydrochloride (pattern K), hydrochloride hydrate (pattern L), sulfate (pattern M), tetrahydrofuran solvate (pattern N).

The PCT publication WO 2017/058392 A1 describes a solid dispersion comprising an active ingredient (venetoclax).

The PCT publication WO 2017/063572 A1 describes several crystalline forms of venetoclax which are designated as form B, form D, form F and form G.

The process described in the prior art reports crystalline and amorphous solid state forms of venetoclax and the process for preparation of crystalline forms.

There are disclosures in the art for crystalline and amorphous forms of Venetoclax and the processes for the preparation thereof; however, it is known that the amorphous forms of a number of pharmaceutical substances exhibit different dissolution characteristics and in some cases bioavailability patterns compared to crystalline forms. For some therapeutic indications the bioavailability is one of the key parameters determining the form of the substance to be used in a pharmaceutical formulation.

Therefore, there is a constant need for the novel crystalline and amorphous solid state forms and processes thereof. There also is a need of such crystalline forms to enable the preparation of Venetoclax in an amorphous form, wherein any crystalline form, mixture of crystalline forms, mixture of crystalline and amorphous form, solvates or hydrates of Venetoclax can be used as starting material and can be converted into the amorphous form of Venetoclax or amorphous form can be isolated directly from the reaction mixture.

Thus the present invention provides an efficient, economically viable, easily scalable, less hazardous, easy to handle and eco-friendly process for the preparation of venetoclax. The present invention also provides novel crystalline solid state forms and processes thereof. Furthermore, the present invention provides economically viable process for amorphous venetoclax. The solid state forms described herein are useful for therapeutic purposes.

### SUMMARY OF THE INVENTION

The present invention provides novel solid state forms of Venetoclax.

In another embodiment, the solid state forms of Venetoclax of the present invention are crystalline in nature.

In another embodiment, the solid state forms of Venetoclax of the present invention are crystalline solvates.

In yet another embodiment, the present invention provides a process for the preparation of crystalline Venetoclax.

In further embodiment, the present invention provides a process for the preparation of amorphous Venetoclax, comprising of:

a) providing a solution of Venetoclax in a suitable solvent or a mixture of solvents; and

b) isolating amorphous Venetoclax.

In yet further embodiment, the present invention provides a process for the preparation of amorphous Venetoclax from crystalline Venetoclax of present invention.

In one more embodiment, the present invention provides a process for the preparation of venetoclax.

### ABBREVIATIONS

In the context of the present invention the following abbreviations have the indicated meaning, unless explicitly stated otherwise:

XRPD: X-ray powder diffraction pattern/diffractogram

DSC: Differential Scanning Calorimetry

TGA: Thermal Gravimetric Analysis

IR: Infrared spectrum

RH: relative humidity

### BRIEF DESCRIPTION OF THE DRAWINGS

FIG. 1: XRPD of amorphous venetoclax

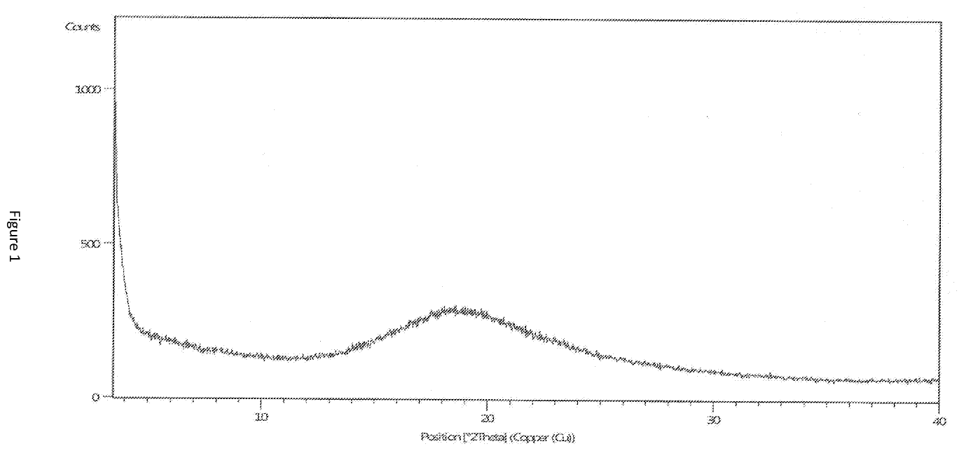

FIG. 2: DSC thermogram of amorphous venetoclax

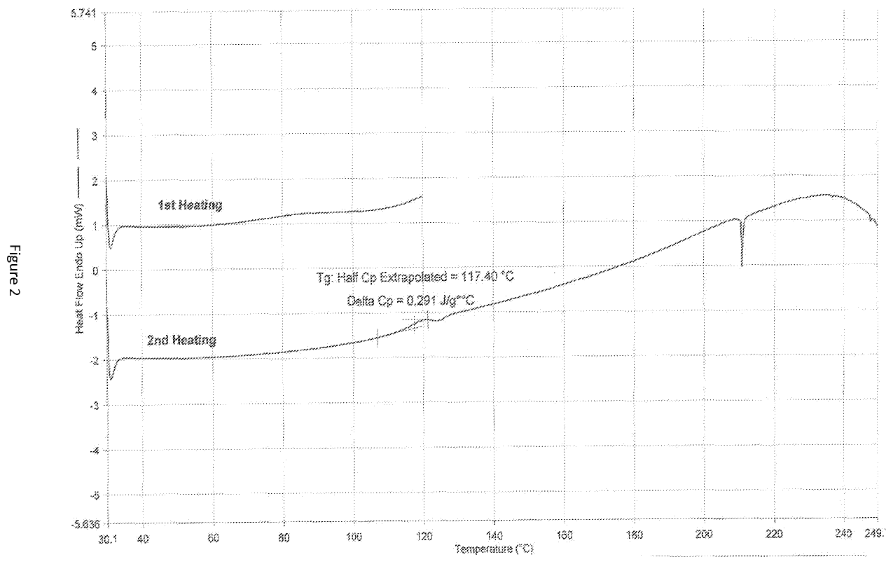

FIG. 3: TGA of amorphous venetoclax

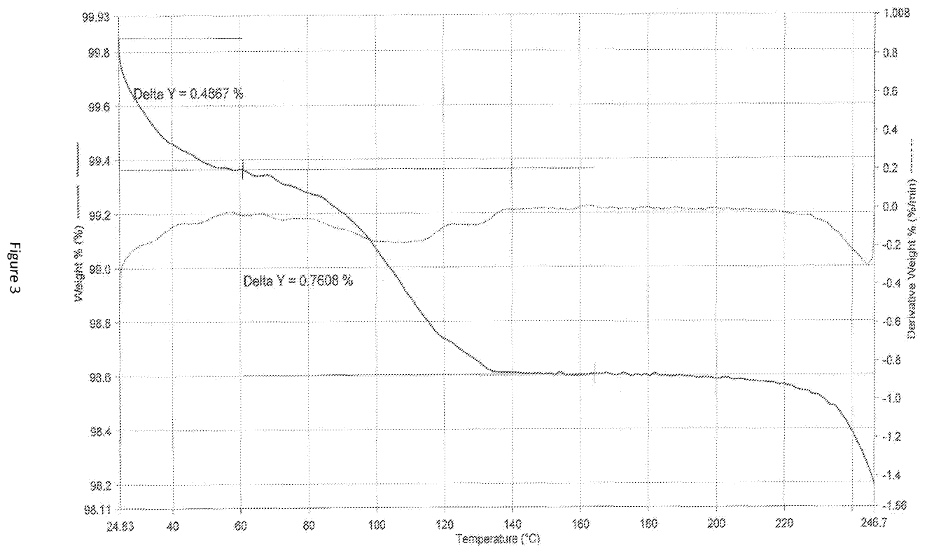

FIG. 4: IR spectrum of amorphous venetoclax

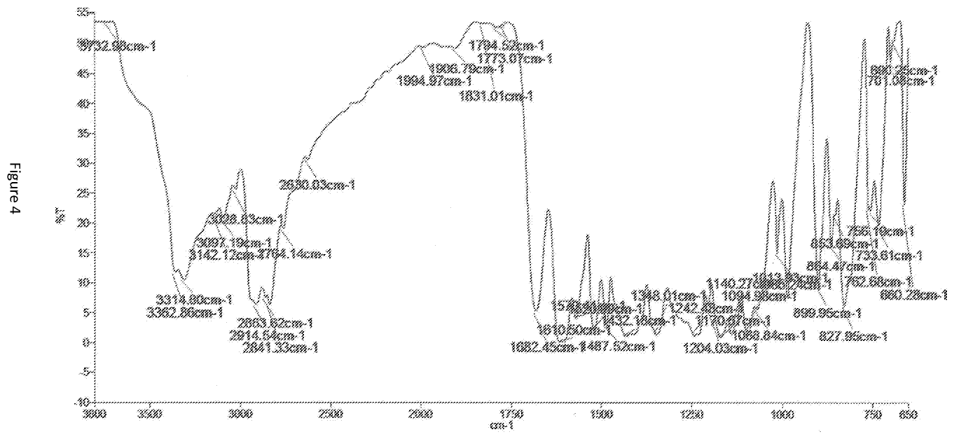

FIG. 5: XRPD of crystalline solid state Form L1 of venetoclax

FIG. 6: DSC thermogram of crystalline solid state Form L1 of venetoclax

FIG. 7: TGA thermogram of crystalline solid state Form L1 of venetoclax

FIG. 8: IR spectrum of crystalline solid state Form L1 of venetoclax

FIG. 9: XRPD of crystalline solid state Form L2 of venetoclax

FIG. 10: DSC thermogram of crystalline solid state Form L2 of venetoclax

FIG. 11: TGA thermogram of crystalline solid state Form L2 of venetoclax

FIG. 12: IR spectrum of crystalline solid state Form L2 of venetoclax

FIG. 13: XRPD of crystalline solid state Form L3 of venetoclax

FIG. 14: DSC thermogram of crystalline solid state Form L3 of venetoclax

FIG. 15: TGA thermogram of crystalline solid state Form L3 of venetoclax

FIG. 16: XRPD of crystalline solid state Form L4 of venetoclax

FIG. 17: DSC thermogram of crystalline solid state Form L4 of venetoclax

FIG. 18: TGA of crystalline solid state Form L4 of venetoclax

FIG. 19: XRPD of amorphous venetoclax prepared from crystalline solid state Form L1 of venetoclax

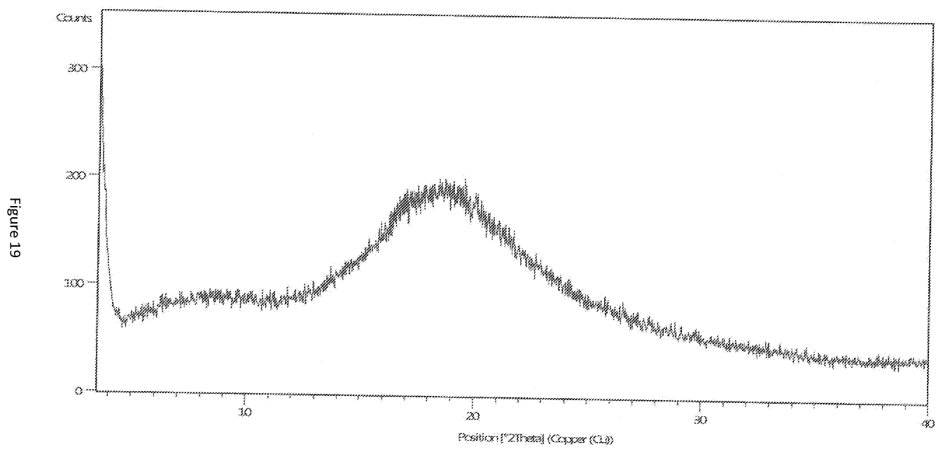

FIG. 20: XRPD of amorphous venetoclax prepared from crystalline solid state Form L2 of venetoclax

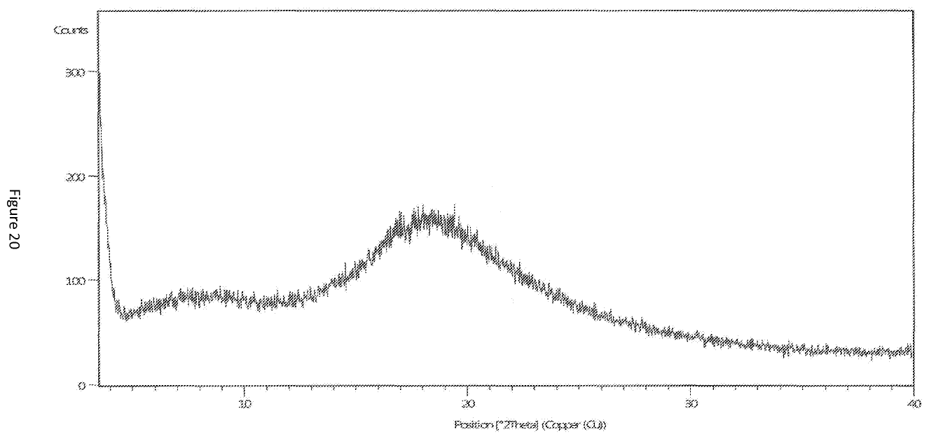

FIG. 21: XRPD of crystalline solid state Form L5 of venetoclax

FIG. 22: IR spectrum of crystalline solid state Form L5 of venetoclax

FIG. 23: XRPD of amorphous venetoclax prepared from crystalline solid state Form L5 of venetoclax

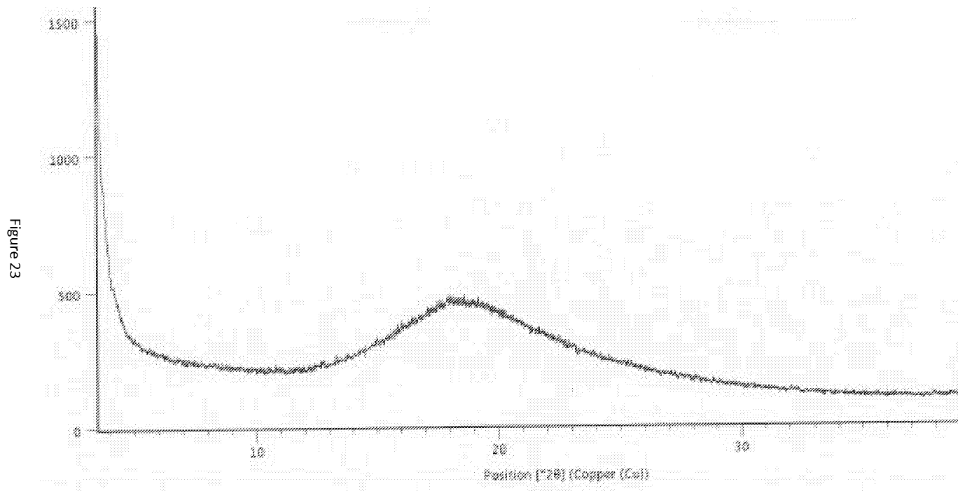

FIG. 24: IR spectrum of amorphous venetoclax prepared from crystalline solid state Form L5 of venetoclax

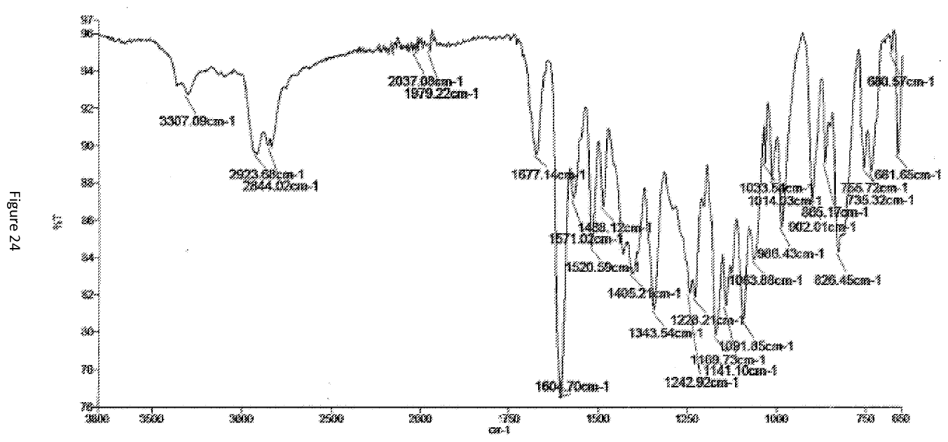

### DETAILED DESCRIPTION OF THE INVENTION

As used herein, the term “amorphous” refers to a solid without definite structure, i.e., lacking crystalline structure.

As used herein, the term “essentially free of crystalline forms” means that no crystalline solid state forms can be detected within the limits of an X-ray Powder Diffractometer by methods known to those skilled in the art at the time of the filing of this application.

As used herein, the term “stable” includes either: amorphous venetoclax that after exposure to a relative humidity of 60% at 25° C. or 75% at 40° C., for a period of at least three months does not convert to any other polymorphic forms.

“Substantially as depicted in” in the context of the present invention means a pattern that is not necessarily identical to those depicted herein, but falls within the limits of experimental or statistical error or deviations when considered by the person skilled in the art.

The term “solvate” as used herein and unless indicated otherwise, refers to a crystal form that incorporates a solvent in the crystal structure. The solvent in a solvate may be present in either a stoichiometric or in a non-stoichiometric amount.

Accordingly, the present invention provides a crystalline solid state selected from Form L1, Form L2, Form L3, Form L4 and Form L5 of venetoclax.

In another embodiment, the present invention provides a crystalline solid state Form L1 of venetoclax.

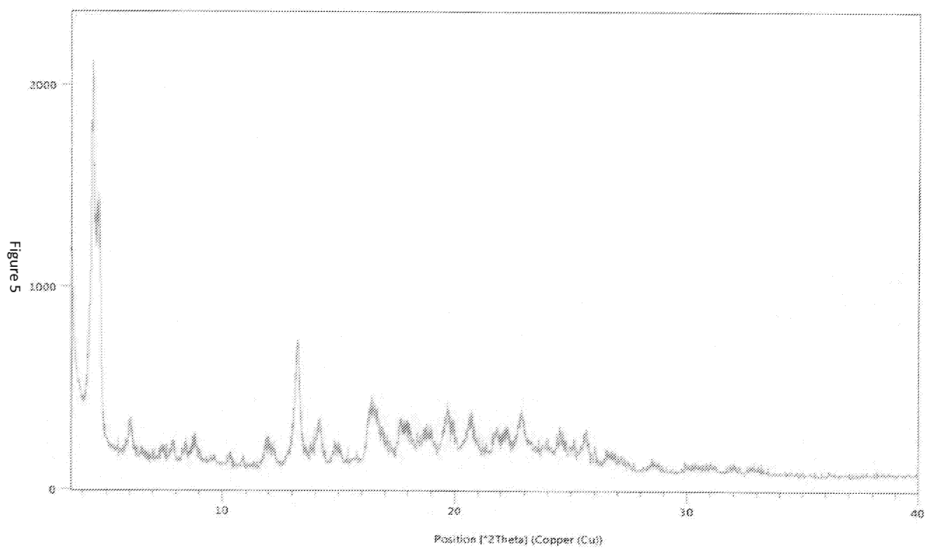

Preferably, crystalline solid state Form L1 of venetoclax of the present invention is characterized by an X-ray powder diffraction pattern with characteristic peaks at 4.4, 4.6, 13.2±0.2° 2θ when measured at a temperature of 25° C. with Cu K-alpha radiation (FIG. 5).

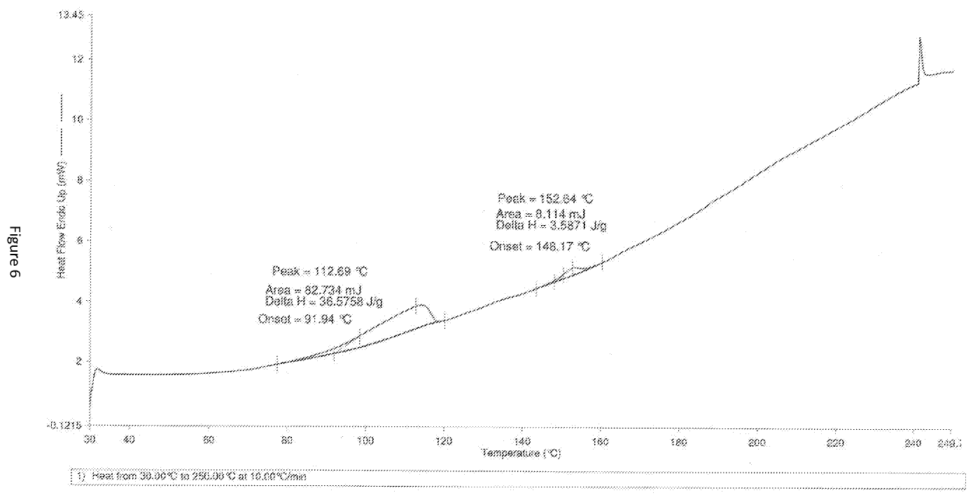

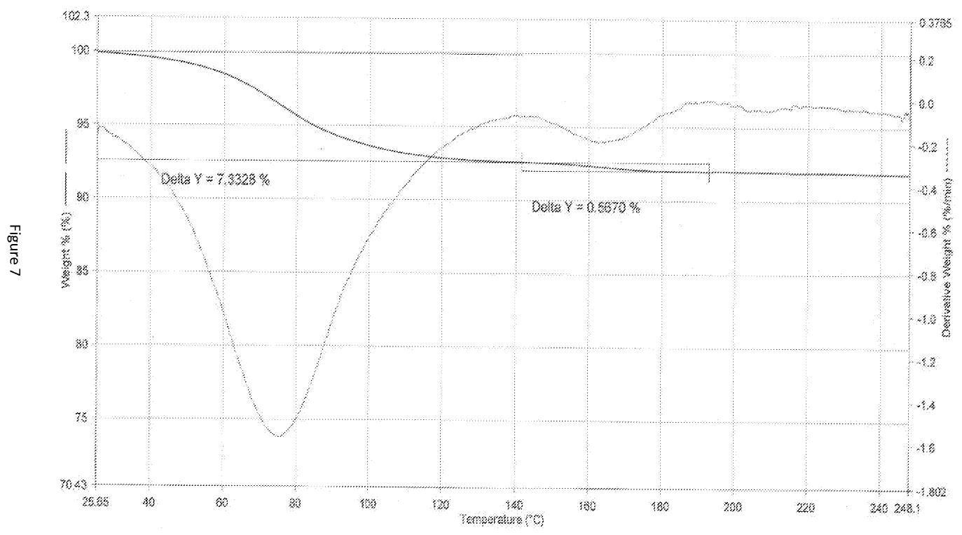

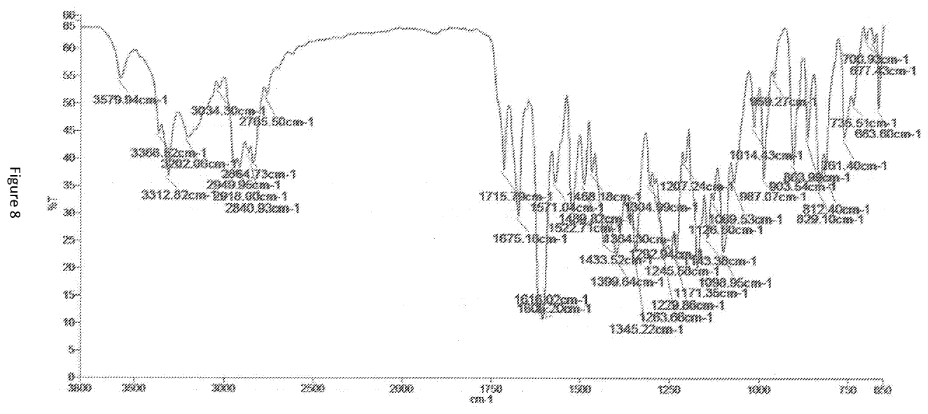

Additionally, crystalline solid state Form L1 of venetoclax is characterized by Differential Scanning Thermogram (DSC), Thermogravimetric Analysis (TGA) and Infrared (IR) spectrum substantially as depicted in FIG. 6, FIG. 7 and FIG. 8 respectively.

In another embodiment of the present invention, the crystalline solid state Form L1 of venetoclax is a crystalline isopropyl acetate solvate.

The present invention also provides a crystalline solid state form L2 of venetoclax.

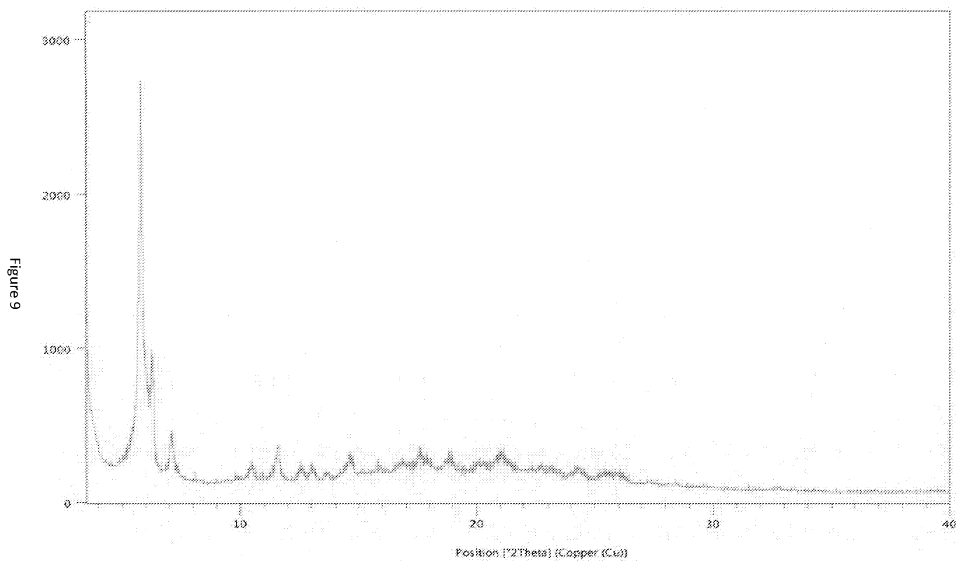

Preferably, crystalline solid state Form L2 of venetoclax of the present invention is characterized by an X-ray powder diffraction pattern with characteristic peaks at 5.7, 6.2, 7.0 and 11.5±0.2° 2θ when measured at a temperature of 25° C. with Cu K-alpha radiation (FIG. 9).

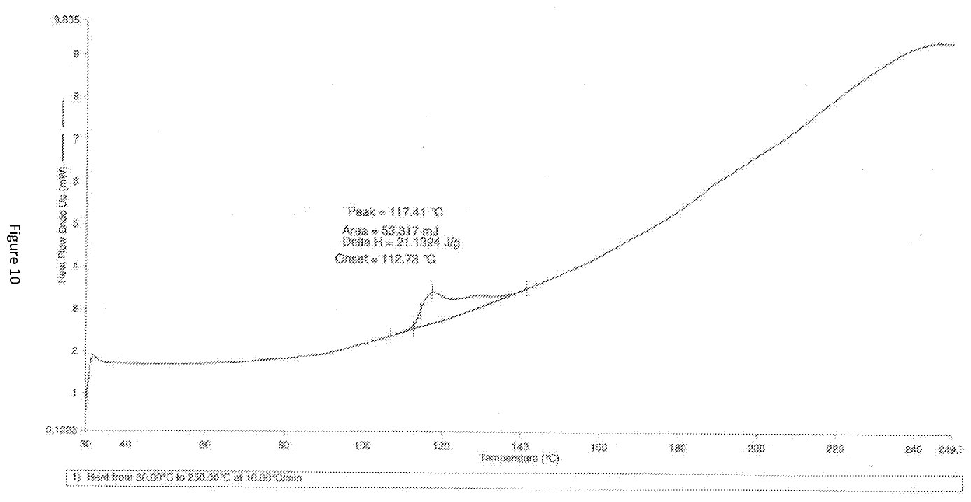

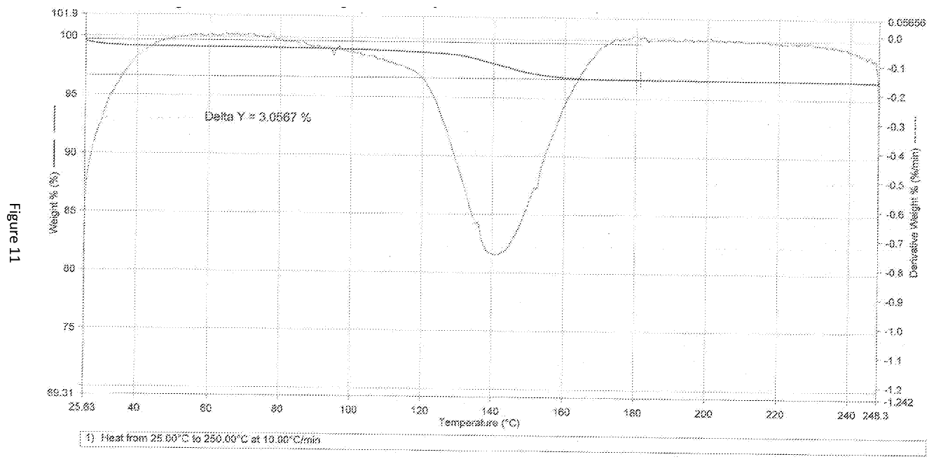

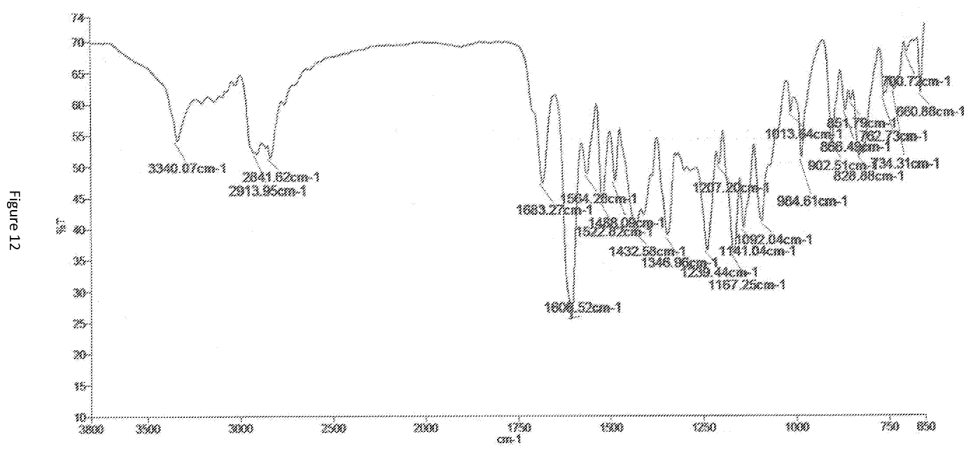

Additionally, crystalline solid state Form L2 of venetoclax is characterized by Differential Scanning Thermogram (DSC), Thermogravimetric Analysis (TGA) and Infrared (IR) spectrum substantially as depicted in FIG. 10, FIG. 11 and FIG. 12 respectively.

In another embodiment of the present invention, the crystalline solid state Form L2 of venetoclax is a crystalline methyl ethyl ketone solvate.

The present invention also provides a crystalline solid state Form L3 of venetoclax.

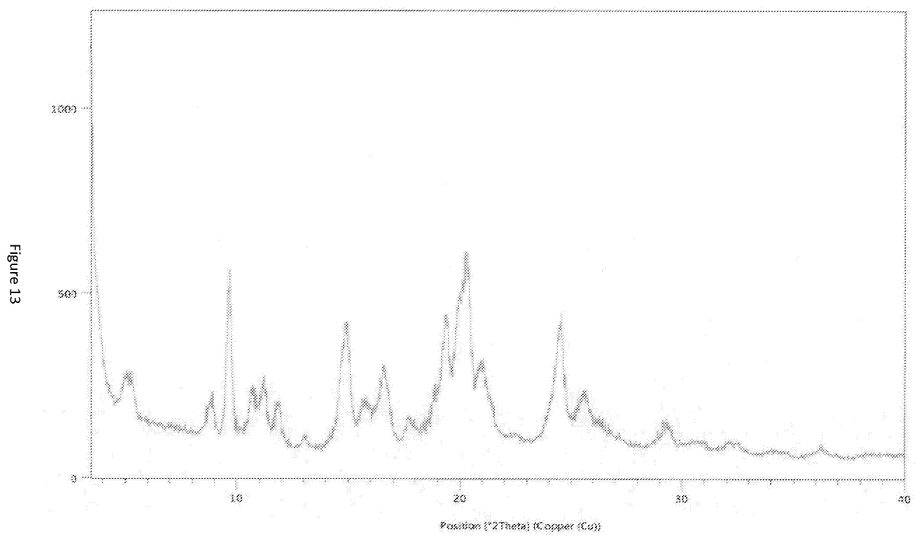

Preferably, crystalline solid state Form L3 of venetoclax of the present invention is characterized by an X-ray powder diffraction pattern with characteristic peaks at 4.8, 9.6, 11.2, 14.6, 19.9 and 24.5±0.2° 2θ when measured at a temperature of 25° C. with Cu K-alpha radiation (FIG. 13).

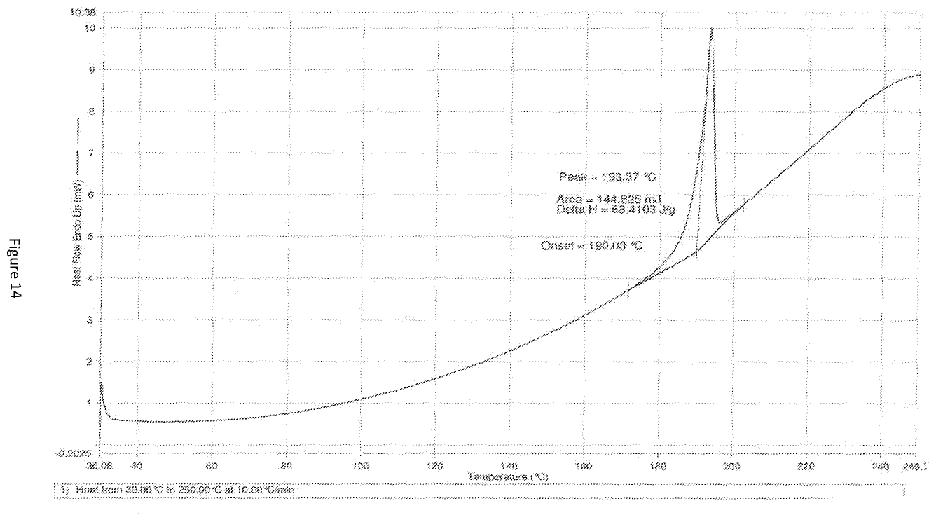

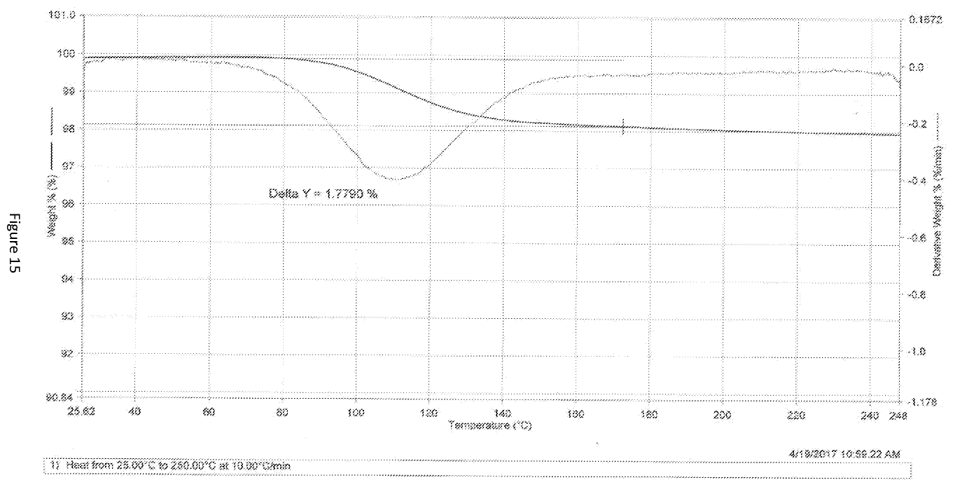

Additionally, crystalline solid state Form L3 of venetoclax is characterized by Differential Scanning Thermogram (DSC) and Thermogravimetric Analysis (TGA) substantially as depicted in FIG. 14 and FIG. 15.

In another embodiment of the present invention, the crystalline solid state Form L3 of venetoclax is a crystalline toluene solvate.

The present invention also provides a crystalline solid state form L4 of venetoclax.

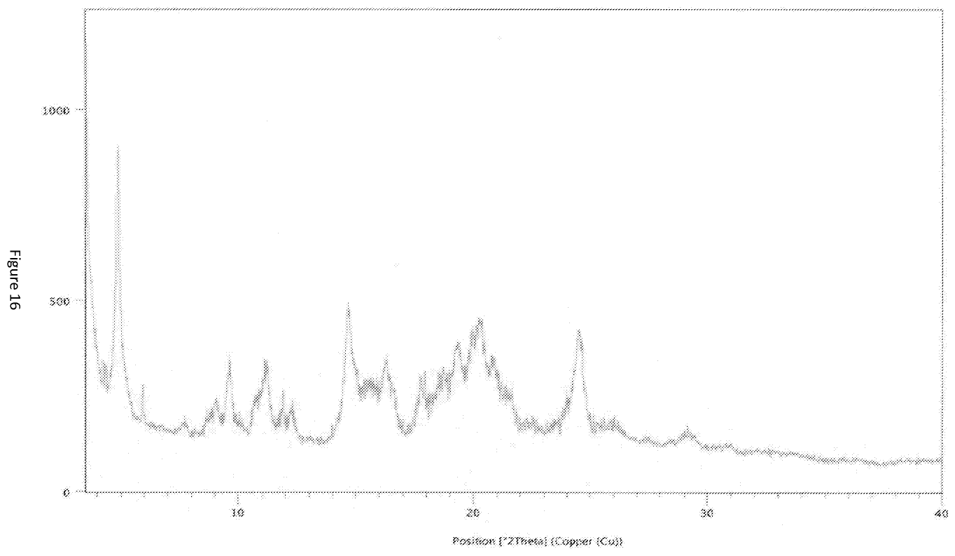

Preferably, crystalline solid state Form L4 of venetoclax of the present invention is characterized by an X-ray powder diffraction pattern with characteristic peaks at 9.6, 14.9, 19.3, 19.9, 20.2 and 24.5±0.2° 2θ when measured at a temperature of 25° C. with Cu K-alpha radiation (FIG. 16).

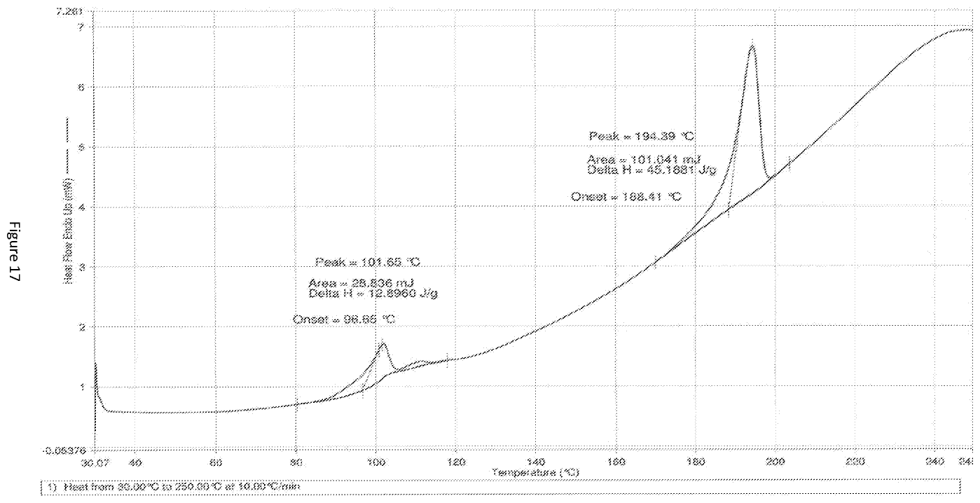

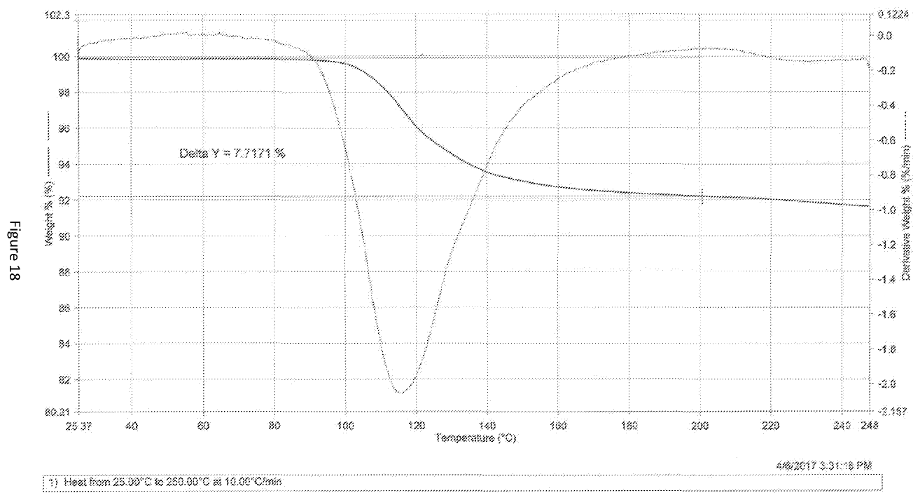

Additionally, crystalline solid state Form L4 of venetoclax is characterized by Differential Scanning Thermogram (DSC) and Thermogravimetric Analysis (TGA) substantially as depicted in FIG. 17 and FIG. 18 respectively.

In another embodiment of the present invention, the crystalline solid state Form L4 of venetoclax is a crystalline anisole solvate.

The present invention also provides a crystalline solid state Form L5 of venetoclax.

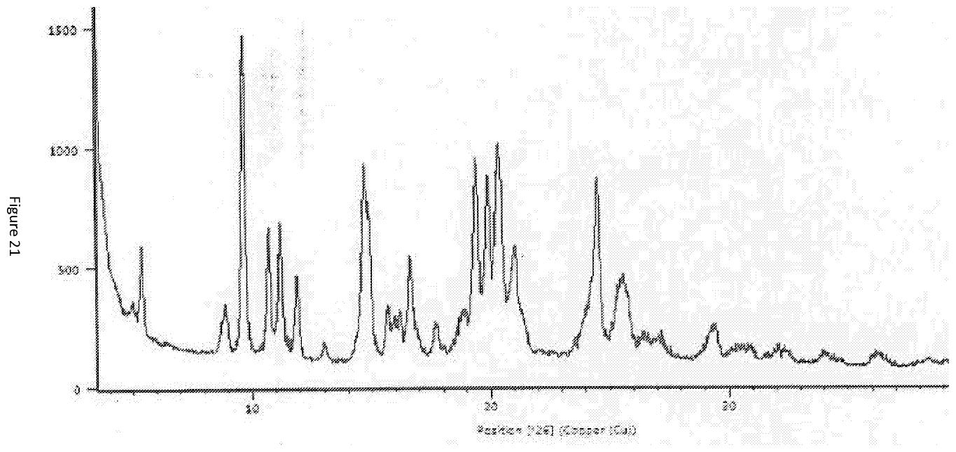

The crystalline solid state Form L5 of venetoclax of the present invention is characterized by an X-ray powder diffraction pattern with characteristic peaks at 9.6, 14.7, 19.4, 19.9, 20.3, 24.5±0.2° 2θ when measured at a temperature of 25° C. with Cu K-alpha radiation (FIG. 21).

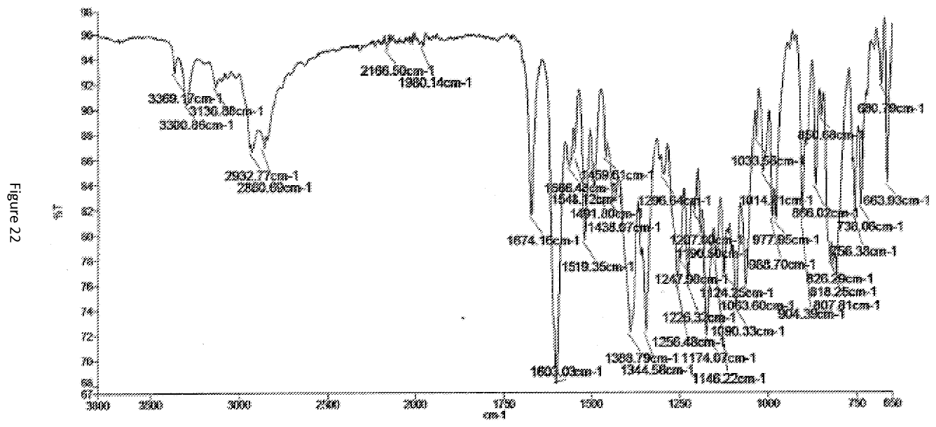

Additionally, crystalline solid state Form L5 of venetoclax is characterized by Infrared (IR) spectrum substantially as depicted in FIG. 22.

In another embodiment of the present invention, the crystalline solid state Form L5 of venetoclax is a crystalline xylene solvate. In a further embodiment, the crystalline solid state Form L5 of venetoclax is a crystalline o-xylene solvate.

In another embodiment, the present invention provides a process for the preparation of novel crystalline solid state forms L1, L2, L3, L4 and L5 of venetoclax comprising:

a) providing a solution of venetoclax in a suitable solvent, and

b) isolating the suitable crystalline solid state Form L1, Form L2, Form L3, Form L4 or Form L5 of venetoclax from the solution.

The suitable solvent used herein for making the crystalline solid state Form L1, Form L2, Form L3, Form L4 or Form L5 of venetoclax is selected form isopropyl acetate, methyl ethyl ketone, toluene, anisole, and xylene.

The solution of venetoclax may be obtained by dissolving venetoclax in a solvent mentioned herein. The dissolution temperature to prepare the solution of venetoclax can range from about 10° C. to reflux temperature of solvent. The obtained solution can be optionally treated with carbon for removal of undesired color or filtered for removal of any undissolved particles. The above solution is then cooled, preferably cooled to a temperature of 20-25° C., and the corresponding crystalline solid state form obtained is isolated by one or more of washing, crystallization, precipitation, cooling, filtration, filtration under vacuum, decantation and centrifugation, or a combination thereof.

Another embodiment of the present invention provides use of novel crystalline solid state forms of venetoclax of the present invention for the manufacture of amorphous venetoclax. In particular, it has surprisingly been found in the present invention that amorphous venetoclax can be prepared by using the novel crystalline solid state forms L1, L2, L3, L4, and L5 of venetoclax of the present invention. Drying or heating the novel crystalline solid state forms of venetoclax of the present invention at a temperature preferably less than 150° C., preferably between 40 and 130° C. from about 10 minutes to about 24 hours under atmospheric pressure or reduced pressure, gives the amorphous venetoclax.

Thus, the present invention provides a process for the preparation of amorphous venetoclax, comprising:

a) providing a solution of crystalline solid state forms L1, L2, L3, L4, and L5 of venetoclax in a suitable solvent or a mixture of solvents, specifically in dimethyl acetamide;

b) optionally adding an anti-solvent to the solution provided in step (a); and

c) isolating amorphous venetoclax.

The solution of venetoclax may be obtained by dissolving venetoclax in a suitable solvent or a mixture of solvents, or such a solution may be obtained directly from a reaction in which venetoclax is formed.

Suitable solvents for dissolving venetoclax include but are not limited to: alcoholic solvents such as methanol, ethanol, isopropanol, and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, diisopropyl ether, 1,4-dioxane, tetrahydrofuran and the like; hydrocarbons such as toluene, xylene, n-heptane, cyclohexane, n-hexane and the like; nitriles such as acetonitrile, propionitrile and the like; N-Methyl-2-pyrrolidone, dimethyl formamide, dimethyl acetamide and dimethyl sulfoxide, and any mixtures thereof.

Optionally, an anti-solvent is added to the solution of venetoclax obtained hereinabove.

The dissolution temperature to prepare the solution of venetoclax can range from about 10° C. to reflux temperature of solvent, or any other suitable temperatures, as long as a clear solution of venetoclax is obtained.

The quantity of solvent used for dissolution depends on the solvent and the dissolution temperature adopted. The concentration of venetoclax in the solution may range from about 0.1 to about 5 g/ml in the solvent, and the volume of the solvent may be kept to a minimum so as to facilitate the effective solvent removal.

The venetoclax solution obtained above can be optionally treated with one or more of carbon, flux-calcined diatomaceous earth (Hyflow) or any other suitable material for removal of undesired color, improve clarity of the solution, and/or remove impurities adsorbable on such material, or a combination thereof.

The venetoclax solution obtained above may be treated for removal of any undissolved particles by subjecting the solution to one or more of filtration, centrifugation, decantation, and other techniques known in the art, or a combination thereof.

In yet another embodiment, isolation of amorphous venetoclax may be effected by combining the solution obtained in step a) with a suitable anti-solvent. Anti-solvent as used herein refers to a liquid in which venetoclax is less soluble or poorly soluble.

Suitable anti-solvent is selected from water, water-miscible organic solvent or any mixtures thereof. Water-miscible organic solvent includes but not limited to acetone, methanol, ethanol, isopropanol, dioxane, acetonitrile and the like.

The ratio of solvent to anti-solvent can be any useful ratio. In some embodiments, the ratio of solvent to anti-solvent is from about 1:1 to about 1:25 (vol/vol), preferably about 1:20 (vol/vol), more preferably about 1:10 (vol/vol).

In another embodiment, the process of step c) involves isolating amorphous venetoclax from the solution obtained. Isolation of amorphous venetoclax in step c) may involve one or more methods including removal of solvent, cooling, crash cooling, distillation, concentrating the mass, evaporation, flash evaporation, simple evaporation, rotational drying, spray drying, thin-film drying, agitated thin film drying, agitated nutsche filter drying, pressure nutsche filter drying, freeze-drying, lyophilization, adding anti-solvent or any other suitable technique, or a combination thereof.

The solid residue of venetoclax obtained after contacting with anti-solvent can be isolated by using various filtration techniques such as Nutsche filter, decantation, centrifugation, gravity filtration, membrane filtration, or suction filtration, or a combination thereof.

Suitable temperatures for isolation may be less than about 120° C., less than about 80° C., less than about 60° C., less than about 40° C., less than about 30° C., less than about 20° C., less than about 10° C., less than about 0° C., less than about −10° C., less than about −40° C. or any other suitable temperatures as long as venetoclax is obtained without affecting its quality.

Another embodiment of the present invention provides a process for preparation of amorphous venetoclax comprising melt crystallization technique.

The amorphous or crystalline solid state forms of venetoclax of the present invention may optionally be further dried. Drying may be carried out using conventional methods such as tray drier, vacuum oven, air oven, cone vacuum dryer, rotary vacuum dryer, fluidized bed dryer, spin flash dryer, flash dryer, or the like. The drying may be carried out at temperatures of less than about 60° C., less than about 50° C., less than about 40° C., less than about 30° C., less than about 20° C., or any other suitable temperatures; at atmospheric pressure or under a reduced pressure; as long as the venetoclax is not degraded in its quality. The drying can be carried out for any desired times until the required product quality is achieved. Suitable time for drying can vary from few minutes to several hours for example from about 30 minutes to about 24 or more hours.

The amorphous or crystalline solid state forms of venetoclax of the present invention may be micronized to achieve the better particle size distribution in order to make suitable Formulation. Micronization may be performed prior to drying or after the drying, by using one or a combination of ball milling, jet milling, jet blending, high-pressure homogenation, or any other conventional milling method.

The amorphous or crystalline solid state forms of venetoclax of the present invention is having a purity of greater than about 95%, or greater than about 98%, or greater than about 99%, or greater than about 99.5%, or greater than about 99.8%, or greater than about 99.9%, as determined using high performance liquid chromatography (HPLC).

The amorphous or crystalline solid state form of venetoclax of the present invention is suitable for pharmaceutical use and having greater stability and is essentially free of crystalline forms.

Venetoclax used for the preparation of the crystalline solid state forms L1, L2, L3, L4, and L5 can be prepared by any process known in the art or by any novel process. In a preferred embodiment of the present invention, a process involving the intermediate compounds of Formula (V) and (VI) is described below.

Accordingly, the present invention provides a process for the preparation of venetoclax comprising,
i) Treating a compound of formula (II)

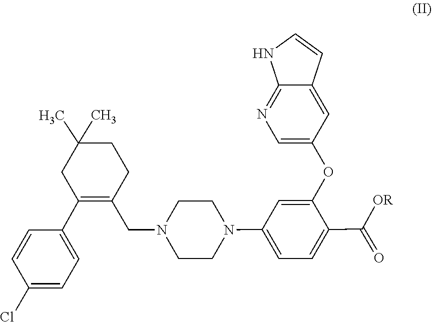

with a base to provide a compound of formula (V)

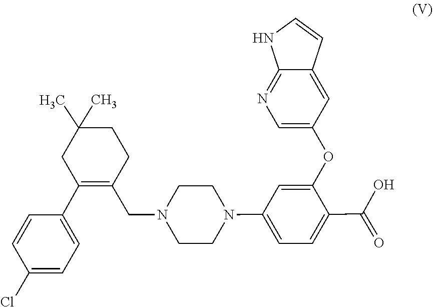 [SMILES: CC1(C)CCC(CN2CCN(c3ccc(C(=O)O)c(Oc4cnc5[nH]ccc5c4)c3)CC2)=C(c2ccc(Cl)cc2)C1]

ii) combining the compound of formula (V) with a compound of formula (VI)

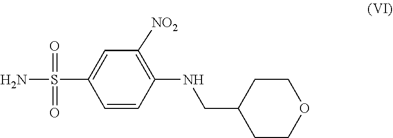 [SMILES: NS(=O)(=O)c1ccc(NCC2CCOCC2)c([N+](=O)[O-])c1]

in presence of a coupling agent, optionally in presence of a base.

Base used is organic or inorganic, wherein, organic base is selected from pyridine, diethylamine, pyrolidine, 4-Dimethylaminopyridine (DMAP), dibutyl amine, triethyl amine (TEA), N,N-Diisopropylethylamine (DIPEA), tributyl amine, 1,4-diazabicycloundec-7-ene (DBU), 1,5-diazabicyclo[4.3.0]non-5-ene (DBN), 1,4-diazabicyclo[2.2.2]-octane (DABCO), diminazen, benzamidine, phosphazenes, amidines, guanidines, and mixtures thereof.

Inorganic base is selected from sodium hydroxide, potassium hydroxide, lithium hydroxide, calcium hydroxide, sodium carbonate, sodium bicarbonate, potassium carbonate, potassium bicarbonate, sodium methoxide, potassium methoxide, sodium hydride, potassium hydride, and mixtures thereof.

Suitable solvents used in the process include, for example, water, acetonitrile, propionitrile, tetrahydrofuran (THF), 1,4-dioxane, diethyl ether, diisopropyl ether, methanol, ethanol, propanol, acetone, methyl ethyl ketone, dimethylformamide, dimethylacetamide (DMAC), dimethyl sulfoxide (DMSO), N-Methyl-2-pyrrolidone, ethyl acetate, butyl acetate, dichloromethane, chloroform, benzene, toluene, xylene or mixtures thereof.

Examples of coupling agents used in the process include I-ethyl-3-[3-(dimethylamino)propyl]-carbodiimide hydrochloride (EDAC), 1,1′-carbonyldiimidazole (CDI), N,N′-Dicyclohexylcarbodiimide (DCC), N,N′-diisopropylcarbodiimide (DIC), 3-(diethoxyphosphoryloxy)-1,2,3-benzotriazin-4(3H)-one (DEPBT), 2-(1H-benzotriazol-1-yl)-1,1,3,3-tetramethyluronium hexafluorophosphate (HBTU), 1-[Bis(dimethylamino)methylene]-1H-1,2,3-triazolo[4,5-b]pyridinium 3-oxid hexafluorophosphate (HATU) and benzotriazol-1-yl-oxytripyrrolidinophosphonium hexafluorophosphate (PyBOP).

The compound of formula (III), compound of formula (IV) and compound of formula (VI) used for the process of the present invention can be prepared by any of the methods known in the prior art. The compound of the formula (III) is used as such or in the form of its hydrochloride salt.

The intermediates or products obtained by any of the processes provided herein may be isolated or recovered by conventional means, such as filtration, concentration, evaporation, extraction, etc. and may be purified by standard procedures, such as distillation, recrystallization or chromatography.

Optionally the compound of formula (I) is purified by treating with xylene, preferably with o-xylene, to furnish a product compatible to the internationally acceptable norms of quality.

The compound of formula (I) obtained by the process of the present invention is highly pure having HPLC purity greater than 98.9%, and is therefore suitable for use as an API for the preparation of a pharmaceutical compositions ready for administration.

In another embodiment, the compound of formula (II)

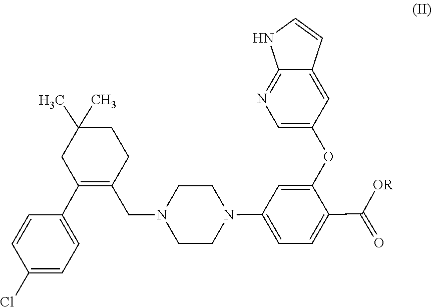

wherein R is C1 to C₄ alkyl
   is prepared by a process, which comprises:
(i) combining a compound of formula (III)

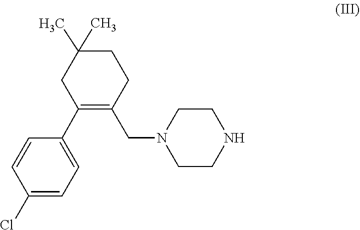 [SMILES: CC1(C)CCC(CN2CCNCC2)=C(c2ccc(Cl)cc2)C1]

with a compound of formula (IV)

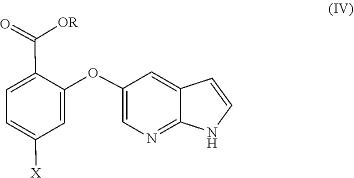

wherein X is halogen and R is C₁ to C₄ alkyl in presence of an organic base.

R is C₁ to C₄ alkyl is selected from the group comprising of methyl, ethyl, n-propyl, isopropyl, n-butyl, tert-butyl, iso-butyl and neo-butyl.

X is halogen is selected from fluorine, chlorine and bromine.

Suitable organic bases include, for example, pyridine, diethylamine, pyrolidine, 4-dimethylaminopyridine (DMAP), dibutyl amine, triethyl amine (TEA), N,N-diisopropylethylamine (DIPEA), tributyl amine, 1,4-diazabicycloundec-7-ene (DBU), 1,5-diazabicyclo[4.3.0]non-5-ene (DBN), 1,4-diazabicyclo[2.2.2]-octane (DABCO), diminazen, benzamidine, phosphazenes, amidines, guanidines, and mixtures thereof.

Suitable solvents include, for example, ethanol, methanol, propanol, acetone, methyl ethyl ketone, tetrahydrofuran (THF), ethyl acetate, dimethyl sulfoxide (DMSO), acetonitrile, dimethylformamide, 1,4-dioxane, chloroform, dichloromethane, toluene, xylene or mixtures thereof.

The reaction is carried out at a temperature of 40-100° C. In this temperature range, it is possible to maintain a stable homogeneous reaction system, with a total reaction time suitable for industrial production, while maintaining a low impurity profile.

The intermediate of formula (II) obtained by the process of the present invention is having HPLC purity greater than 95% and is obtained in greater than 60% in yield.

The intermediate compound of formula (II) obtained by the process of the present invention can be converted to venetoclax by the methods known in the art.

The amorphous venetoclax or crystalline solid state forms of venetoclax of the present invention can be formulated into various pharmaceutical compositions comprising therapeutically effective amount of an amorphous venetoclax or crystalline solvates of venetoclax and one or more pharmaceutically acceptable carriers, excipients or diluents.

The pharmaceutical composition can be solid oral dosage forms such as powders, granules, pellets, tablets and capsules, pills, suppositories, sachets, troches or lozenges; liquid oral dosage forms such as syrups, suspensions, dispersions, emulsions; and injectables.

To understand the present invention following preparative and testing examples are set forth, which are for the purposes of illustration only and are not to be construed as limiting the scope of the invention in any way.

### EXAMPLES

### Example 1: Preparation of Intermediate (II) [R═CH3:2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl) piperazin-1-yl)benzoic Acid Methyl Ester]

In round bottom flask equipped with mechanical stirrer and thermometer, to dimethylsulfoxide (125 ml), pyrrolopyridine ester (IV; X═F & R═CH3) (25 g) and piperazine dihydrochloride (III) (45 g) were added. To this reaction mixture, 1,8-diazabicyclo[5.4.0]undec-7-ene (DBU) (79 ml) was added and the reaction was heated to 75-85° C. and maintained for 46 hrs. Reaction mixture was then cooled to room temperature and ice cooled water (1000 ml) was added. Solid separated was filtered, washed with water and then dissolved in ethyl acetate (500 ml). Ethyl acetate layer was evaporated to obtain gummy mass. To this gummy mass was added isopropyl alcohol (100 ml), stirred and gradually cooled to room temperature to obtain the crude title compound which was recrystallized from isopropyl alcohol to give title compound (33 g).

### Example 2: Preparation of Intermediate (II) [R═CH3:2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl) piperazin-1-yl)benzoic Acid Methyl Ester]

In round bottom flask equipped with mechanical stirrer and thermometer, to dimethylsulfoxide (250 ml), pyrrolopyridine ester (IV; X═F & R═CH3) (50 g) and piperazine dihydrochloride (III) (89 g) were added. To this reaction mixture, 1,8-diazabicyclo[5.4.0]undec-7-ene (DBU) (157 ml) was added and the reaction mixture was heated to 75-85° C. and maintained for 48 hrs. The reaction mixture was then cooled to room temperature and ice cooled water (1000 ml) was added. Solid separated was filtered, washed with water and then dissolved in ethyl acetate (500 ml). To the ethyl acetate layer was added water (500 ml) and ethyl acetate (500 ml), organic layer was extracted, washed with water, dried and concentrated to obtain gummy mass. To this gummy mass was added isopropyl alcohol (200 ml), stirred and filtered to obtain the crude title compound which was recrystallized from isopropyl alcohol to give the pure title compound (47 g).

### Example 3: Preparation of Compound (V) [2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic Acid]

In round bottom flask equipped with mechanical stirrer and thermometer, intermediate (II) (R═CH3) (45 g), dioxane (450 ml) and aq. NaOH solution (9 g in 450 ml water) were stirred at 55° C. for 18 hrs. The reaction was cooled to room temperature, and the reaction mass was concentrated and 6M HCl was added to residue till acidic. The obtained solid was filtered, washed with water, and dried to give the title compound (42 g).

### Example 4: Preparation of Venetoclax

In round bottom flask equipped with mechanical stirrer and thermometer, to dichloromethane (400 ml), 3-nitro-4-((tetrahydro-2H-pyran-4-yl)methylamino)benzene sulfonamide (VI) (17.6 g), 4-dimethylaminopyridine (DMAP) (4.25 g) and I-ethyl-3-[3-(dimethylamino)propyl]-carbodiimide hydrochloride (EDC.HCl) (20.12 g) were added. To this mixture was added a solution of intermediate (V) (40 g) in dichloromethane (400 ml) and trimethyl amine (18.8 ml) and maintained for 22 hrs. To this mixture was then added water (400 ml), the dichloromethane layer was separated, washed with water and concentrated under reduced pressure. The residue obtained was purified by using mixture of ethyl acetate and xylene to give venetoclax (31 g).

### Example 5: Purification of Venetoclax

In round bottom flask equipped with mechanical stirrer and thermometer, charged crude venetoclax (10 g) in o-xylene (200 ml) and stirred at 70-80° C. for 3-4 hrs. The reaction was cooled to 25-30° C., the product obtained was filtered, washed with xylene and dried under reduced pressure to give pure venetoclax (7.3 g).

### Example 6: Preparation of Crystalline Solid State Form L1 of Venetoclax

In round bottom flask equipped with mechanical stirrer, thermometer and an addition funnel, venetoclax (1 g) and isopropyl acetate (15 ml) were heated for 10-30 minutes at 70-80° C. to get clear solution. The solution was cooled to 20-30° C. and kept for evaporation at same temperature. The crystalline material was filtered and dried at 20-30° C. for 10-20 minutes to yield crystalline solid state Form L1 of venetoclax (0.8 g).

### Example 7: Preparation of Crystalline Solid State Form L2 of Venetoclax

In round bottom flask equipped with mechanical stirrer, thermometer and an addition funnel, venetoclax (1 g) and methyl ethyl ketone (10 ml) were heated for 10-30 minutes at 70-80° C. to get clear solution. The solution was cooled to 20-30° C. and kept for evaporation at same temperature. The crystalline material was filtered and dried to yield crystalline solid state Form L2 of venetoclax (0.5 g).

### Example 8: Preparation of Crystalline Solid State Form L3 of Venetoclax

In round bottom flask equipped with mechanical stirrer, thermometer and an addition funnel, venetoclax (4 g) and acetone (80 ml) were heated for 10-30 minutes at 40-60° C. to get clear solution. The solution was distilled and the solid obtained (2.8 g) was leached in 90 ml toluene with stirring, filtered, and dried to yield crystalline solid state Form L3 of venetoclax.

### Example 9: Preparation of Crystalline Solid State Form L4 of Venetoclax

In round bottom flask equipped with mechanical stirrer, thermometer and an addition funnel, venetoclax (1 g) and anisole (5 ml) were heated for 10-30 minutes at 60-70° C. to get clear solution. The solution was filtered and cooled to 20-30° C. and kept for evaporation at same temperature. The crystalline material was filtered and dried to yield crystalline solid state Form L4 of venetoclax (0.3 g).

### Example 10: Preparation of Crystalline Solid State Form L5 of Venetoclax

In round bottom flask equipped with mechanical stirrer and thermometer, charged 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)-piperazin-1-yl)benzoic acid (50 g), dichloromethane (DCM) (375 ml), trimethyl amine (17.3 ml), and I-ethyl-3-[3-(dimethylamino)propyl]-carbodiimide hydrochloride (EDC.HCl) (33.8 g) were added and stirred for 60 min. Above solution was added while stirring in about 90 min to the slurry of 3-nitro-4-((tetrahydro-2H-pyran-4-yl)methylamino)benzene sulfonamide (22 g) in dichloromethane (375 ml) and 4-dimethylaminopyridine (DMAP) (22.6 g). The reaction mixture was stirred for about 6 hrs to get clear solution. The reaction was quenched using 10% acetic acid solution and DCM layer separated after treatment with brine and evaporated in rotavapour. Charged methanol (50 ml) and ethyl acetate (450 ml) slowly to the obtained solid, cooled and stirred at 5-10° C. The resulting slurry was heated to ambient temperature and stirred for 60 min and residual solid dried under vacuum. Charged ethyl acetate (240 ml) and DCM (180 ml) to the solid obtained and stirred and maintained for 16-18 hrs. The reaction mixture was filtered and washed with ethyl acetate and dried. The obtained solid was charged in to o-xylene (1000 ml) and stirred at 75-80° C. for 4-6 hrs. The reaction was further maintained with stirring for 12-15 hrs. The reaction mixture was cooled and the product obtained was filtered, washed with o-xylene and dried under reduced pressure at 75° C. to yield crystalline solid state Form L5 of venetoclax (30 g).

### Example 11: Preparation of Crystalline Solid State Form L5 of Venetoclax

In round bottom flask equipped with mechanical stirrer, thermometer and an addition funnel, charged Venetoclax (2 g) in o-xylene (20 ml) and stirred at 100° C. for 2-3 hrs. The reaction was further maintained with stirring for 12-15 hrs. The reaction mixture was cooled and the product obtained was filtered, washed with o-xylene and dried under reduced pressure at 75° C. to give crystalline solid state Form L5 of venetoclax.

### Example 12: Preparation of Amorphous Venetoclax

In round bottom flask equipped with mechanical stirrer, thermometer and an addition funnel, a solution of venetoclax (20 g) in dimethyl sulfoxide (80 ml) was added slowly with stirring to water (500 ml) at 0-5° C. The reaction mixture was stirred for 10-20 min and then filtered. The solid obtained was washed with 500 ml water and dried under vacuum for 2-3 hrs to yield amorphous venetoclax (17.2 g).

### Example 13: Preparation of Amorphous Venetoclax

In round bottom flask equipped with mechanical stirrer, thermometer and an addition funnel, a solution of venetoclax (3.8 g) in dimethyl acetamide (11.4 ml) was added slowly with stirring to water (114 ml) at 10-15° C. The reaction mixture was stirred for 60 min and then filtered. The solid obtained was washed with water (114 ml) and dried under vacuum to yield amorphous venetoclax (3 g).

### Example 14: Preparation of Amorphous Venetoclax

In round bottom flask equipped with mechanical stirrer, thermometer and an addition funnel, a solution of Venetoclax (5 g) in N-Methyl-2-pyrrolidone (15 ml) was added to water (150 ml) at 5-10° C. The reaction mixture was stirred for 90 min at 10-20° C. and then filtered. The solid obtained was washed with water repeatedly and dried under vacuum to yield amorphous venetoclax (4 g).

### Example 15: Preparation of Amorphous Venetoclax from Crystalline Solid State Form L1 of Venetoclax

Venetoclax isopropyl acetate solvate (0.15 g) was dried in oil bath at 130° C. for 7 hrs to yield amorphous venetoclax (0.11 g).

### Example 16: Preparation of Amorphous Venetoclax from Crystalline Solid State Form L2 of Venetoclax

Venetoclax methyl ethyl ketone solvate (0.2 g) was dried in oil bath at 130° C. for 6.30 hrs to yield amorphous venetoclax (0.15 g).

1. A crystalline solid state form of venetoclax selected from Form L1, Form L2, Form L3, Form L4 and Form L5.

2. A crystalline solid state Form L1 of venetoclax.

3. The crystalline solid state Form L1 of venetoclax of claim 2, characterized by an X-ray powder diffraction pattern having characteristic peaks at 4.4, 4.6, 13.2±0.2° 2θ when measured at a temperature of 25° C. with Cu K-alpha radiation.

4. The crystalline solid state Form L1 of venetoclax of claim 2, wherein said crystalline solid state Form A is crystalline isopropyl acetate solvate.

5. A crystalline solid state Form L2 of venetoclax.

6. The crystalline solid state Form L2 of venetoclax of claim 5, characterized by an X-ray powder diffraction pattern having characteristic peaks at 5.7, 6.2, 7.0 and 11.5±0.2° 2θ when measured at a temperature of 25° C. with Cu K-alpha radiation.

7. The crystalline solid state Form L2 of venetoclax of claim 5, wherein said crystalline solid state Form B is crystalline methyl ethyl ketone solvate.

8. A crystalline solid state Form L3 of venetoclax.

9. The crystalline solid state Form L3 of venetoclax of claim 8, characterized by an X-ray powder diffraction pattern having characteristic peaks at 4.8, 9.6, 11.2, 14.6, 19.9 and 24.5±0.2° 2θ when measured at a temperature of 25° C. with Cu K-alpha radiation.

10. The crystalline solid state Form L3 of venetoclax of claim 8, wherein said crystalline solid state Form C is crystalline toluene solvate.

11. A crystalline solid state Form L4 of venetoclax.

12. The crystalline solid state Form L4 of venetoclax of claim 11, characterized by an X-ray powder diffraction pattern having characteristic peaks at 9.6, 14.9, 19.3, 19.9, 20.2 and 24.5±0.2° 2θ when measured at a temperature of 25° C. with Cu K-alpha radiation.

13. The crystalline solid state Form L4 of venetoclax of claim 11, wherein said crystalline solid state Form D is crystalline anisole solvate.

14. A crystalline solid state Form L5 of venetoclax.

15. The crystalline solid state Form L5 of venetoclax of claim 14, characterized by an X-ray powder diffraction pattern having characteristic peaks at 9.6, 14.7, 19.4, 19.9, 20.3, 24.5±0.2° 2θ when measured at a temperature of 25′C with Cu K-alpha radiation.

16. The crystalline solid state Form L5 of venetociax of claim 14, wherein said crystalline solid state Form E is crystalline o-xylene solvate.

17. A process for the preparation of novel crystalline solid state Form L1, Form L2, Form L3, Form L4 or Form L5 of venetoclax comprising,
a) providing a solution of venetoclax in a suitable solvent, and
b) isolating the suitable crystalline solid state Form L1, Form L2, Form L3, Form L4 or Form L5 of venetoclax from the solution.

18. The process of claim 17, wherein the suitable solvent for making the crystalline solid state Form L1, Form L2, Form L3, Form L4 or Form L5 of venetoclax is selected form isopropyl acetate, methyl ethyl ketone, toluene, anisole, or xylene.

19. A process for the preparation of amorphous venetoclax, comprising:
a) providing a solution of crystalline solid state Form L1, Form L2, Form L3, Form L4 or Form L5 of venetocdax in dimethyl acetamide;
b) optionally adding an anti-solvent to the solution provided in step (a); and
c) isolating amorphous venetoclax.

20. A process for the preparation of venetocdax of formula (I) comprising, 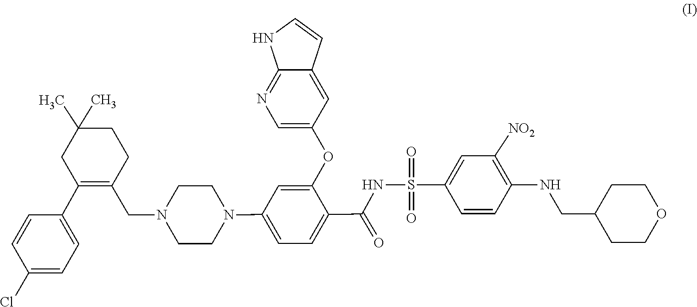 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]
(i) combining a compound of formula (III) 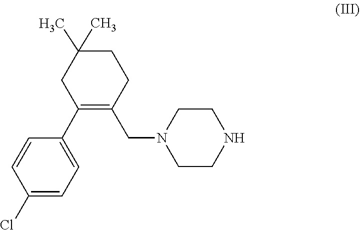 [SMILES: CC1(C)CCC(CN2CCNCC2)=C(c2ccc(Cl)cc2)C1]
with a compound of formula (IV) 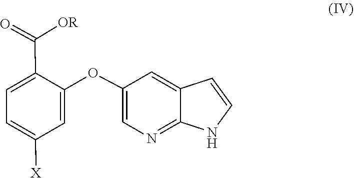
wherein X is halogen and R is C₁ to C₄ alkyl
in presence of an organic base to provide a compound of Formula (II) 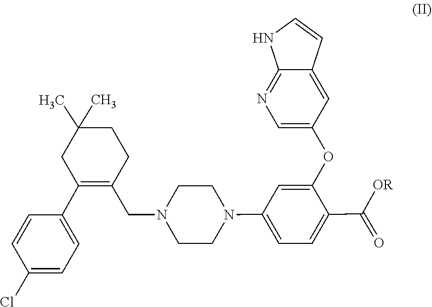
wherein R is C₁ to C₄ alkyl
(ii) treating the compound of formula (II) with a base to provide a compound of formula (v) 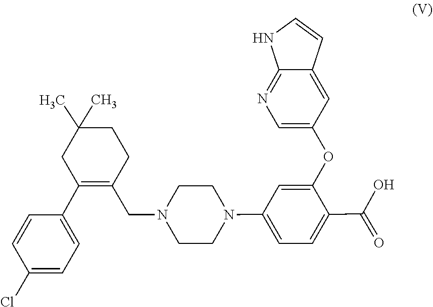 [SMILES: CC1(C)CCC(CN2CCN(c3ccc(C(=O)O)c(Oc4cnc5[nH]ccc5c4)c3)CC2)=C(c2ccc(Cl)cc2)C1]
(iii) combining the compound of formula (V) with a compound of formula (VI) 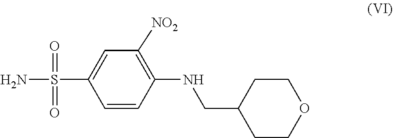 [SMILES: NS(=O)(=O)c1ccc(NCC2CCOCC2)c([N+](=O)[O-])c1]
in presence of a coupling agent, optionally in presence of a base.

21. The process of claim 20, wherein in step (iii) the coupling agent is selected from I-ethyl-3-[3-(dimethylamino)propyl]-carbodiimide hydrochloride; 1,1-carbonyldiimidazole; N,N′-Dicyclohexylcarbodiimide; N,N′-diisopropylcarbodiimide; 3-(diethoxyphosphoryloxy)-1,2,3-benzotriazin-4(3H)-one; 2-(1H-benzotriazol-1-yl)-1,1,3,3-tetramethyluronium hexafluorophosphate; 1-[Bis(dimethylamino)-methylene]-1H-1,2,3-triazolo[4,5-b]pyridinium 3-oxid hexafluorophosphate and benzotriazol-1-yl-oxytripyrrolidinophosphonium hexafluorophosphate.

22. The process of claim 20, wherein in step (iii) the base is an organic base or an inorganic base.

23. The process of claim 22, wherein in step (iii) the base is an organic base selected from pyridine, diethylamine, pyrolidine, 4-Dimethylaminopyridine, dibutyl amine, triethyl amine, N,N-Diisopropylethylamine, tributyl amine, 1,4-diazabicycloundec-7-ene, 1.5-diazabicyclo[4.3.0]non-5-ene, 1,4-diazabicyclo[2.2.2]-octane, diminazen, benzamidine, phosphazenes, amidines, guanidines, and mixtures thereof.

24. The process of claim 22, wherein in step (iii) the base is an inorganic base selected from sodium hydroxide, potassium hydroxide, lithium hydroxide, calcium hydroxide, sodium carbonate, sodium bicarbonate, potassium carbonate, potassium bicarbonate, sodium methoxide, potassium methoxide, sodium hydride, potassium hydride, and mixtures thereof.

25. A process for the preparation of the compound of formula (II) 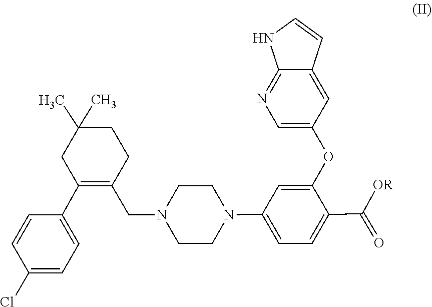
wherein R is C₁ to C₄ alkyl which comprises,
(ii) combining a compound of formula (III) 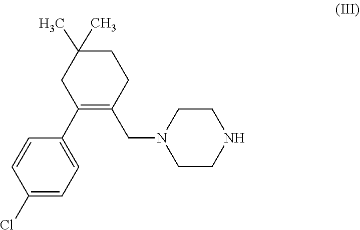 [SMILES: CC1(C)CCC(CN2CCNCC2)=C(c2ccc(Cl)cc2)C1]
with a compound of formula (IV) 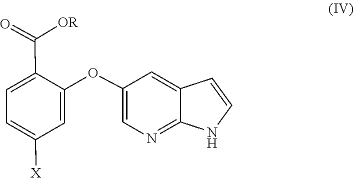
wherein X is a halogen
and R is C₁ to C₄ alkyl
in presence of an organic base.

26. The process of claim 25, wherein the organic base is selected from pyridine, diethylamine, pyrolidine, 4-dimethylaminopyridine, dibutyl amine, triethyl amine, N,N-diisopropylethylamine, tributyl amine, 1,4-diazabicycloundec-7-ene, 1,5-diazabicyclo[4.3.0]non-5-ene, 1,4-diazabicyclo[2.2.2]-octane, diminazen, benzamidine, phosphazenes, amidines, guanidines, and mixtures thereof.

27. The process of claim 25, wherein the compound of formula (II) and the compound of formula (III) are combined at a temperature of 40-100° C.
